(19) United States
(12) Patent Application Publication
(10) Pub. No.: US 2026/0174358 A1
(43) Pub. Date: Jun. 25, 2026

(54) SYSTEM FOR DETERMINING A DISTANCE BETWEEN TWO MOVABLE ELEMENTS BY INDUCTIVE COUPLING AND RELATED METHOD

(71) Applicants: Université de Bordeaux, Bordeaux (FR); TotalEnergies OneTech, Courbevoie (FR); Centre National de la Recherche Scientifique, Paris (FR)

(72) Inventor: Jean-Charles MASSABUAU, Arcachon (FR)

(21) Appl. No.: 19/129,244

(22) PCT Filed: Nov. 14, 2022
(86) PCT No.: PCT/IB2022/000806

§ 371 Date: May 12, 2025

(51) Int. Cl. A61B 5/145; A61N 1/05

(52) U.S. Cl. CPC A61B 5/14532; A61N 1/0551

(57) ABSTRACT

The invention relates to a system (1) for determining a distance between two movable elements, the system comprising a processor, an energy source and a pair of transmitting and receiving electrodes (41, 42), the energy source being configured to generate a first electric current to the transmitting electrode (41) so that the receiving electrode (42) delivers, by inductive coupling, a second electric current, in which: —the electrodes are located at less than 40 mm and include at least: o a magnetic core, o a metallic winding having an inductance ranging from 1 μH to 470 μH, —the processor (10) is configured to convert the electric current delivered by the receiving electrode (42) into an intensity value and to generate a measurement signal including the intensity value, the processor is further configured to carry out a digital processing of the measurement signal to obtain an estimate of the distance.

### FIELD OF THE INVENTION

The invention relates to the field of metrology. It relates to a system for high resolution determination of a distance between two moving elements.

The invention further relates to a method for determining a distance between two moving elements.

### DESCRIPTION OF RELATED ART

Precision micromechanics concentrates a large part of the dimensional measurement problems encountered in lots of sectors of activity: very elaborate parts of small dimensions <5 mm, mirror polished or matt, from very small bores to small tolerances, clumsy shapes that are difficult to access, thin fabrics deforming the part at the slightest contact during measurement.

One of the main challenges is to be provide a very fine measure resolution for a given measuring range in line with measuring principles such as measuring positions, a separation distance, a diameter, and/or an end play.

To that end, the laser micrometer measures without contact can be used in connection with the above-mentioned measuring principles. The universal laser micrometer works on the principle of scanning or shading and is suitable for non-contact measurements of very hot, sticky or sterile materials. This allows it to also be used in automatic production lines. The laser micrometer consists of a sensor unit and a controller. Data from the laser micrometer are sent via an analog or digital port to the measurement system, which allows it to be linked to the manufacturing process.

Whether the laser micrometer is particularly advantageous for individual or serial checks, it is not designed to be integrated in on movable parts or elements of a device or an object.

To allow measuring a distance between movable parts or elements of a device, the Hall effect is mainly used. The Hall effect sensors generally consist of a probe and a magnet. The probe is a semiconductor wafer such as silicon or doped germanium. It is traversed by a current, and the Hall effect sensors is used to measure the electric current intensity at its terminals. The magnet produces an induction field whose value on the probe depends on its position, thus modifying the intensity.

More particularly, one of the possible applications of Hall effect sensors is to measure the opening distance between the two valves of an aquatic invertebrate organism.

Patent document n°WO200210710 discloses a method and a system for monitoring the quality of an environment in real time, based on a Hall effect detection system in order to measure the opening distance between the two valves of an invertebrate aquatic organism. Indeed, depending on the toxicity of an environment, aquatic invertebrate organisms will present an opening rate well different than the one in a healthy environment. However, this system provides a measurement accuracy in the range of 0.5-1 mm and therefore its applications are not adapted to micro distances.

Thus, there is a need for a system that allows to provide micro distances measurement between movable elements with an improved accuracy.

### SUMMARY OF THE INVENTION

The following sets forth a simplified summary of selected aspects, embodiments and examples of the present invention for the purpose of providing a basic understanding of the invention. However, the summary does not constitute an extensive overview of all the aspects, embodiments and examples of the invention. The sole purpose of the summary is to present selected aspects, embodiments and examples of the invention in a concise form as an introduction to the more detailed description of the aspects, embodiments and examples of the invention that follow the summary.

The invention aims to overcome the disadvantages of the prior art. In particular, the invention proposes system for determining a distance between two movable elements, the system comprising at least a processor, an energy source and at least a pair of transmitting and receiving electrodes, the transmitting electrode and the receiving electrode being respectively fixed on the first and the second movable elements, the energy source being further configured to generate a first electric current to the transmitting electrode so that the receiving electrode delivers, by inductive coupling, a second electric current, in which:
the transmitting and the receiving electrodes are located at less than 40 mm, more

Preferably less than 30 mm, from each other and include at least:
a magnetic core,
a metallic winding having an inductance ranging from 1 μH to 470 μH,
the processor is configured to convert the electric current delivered by the receiving electrode, into at least one intensity value and to generate at least one measurement signal which includes said intensity value,
the processor is further configured to carry out a digital processing of the measurement signal to obtain an estimate of the distance between the two movable elements from a mathematical law which links the distance between the two movable elements and the intensity value included in the measurement signal.

The system according to the invention allows to provide high accuracy and high-resolution micro distance measurements. More particularly, the system allows to determine a distance at a measurement accuracy of less than 200 μm and even of less than 5 μm while ensuring a resolution of the measurement of 3-5 μm. The system arrangement is also fitted to be integrated in narrow environment.

According to other optional features of the system according to the invention, it can optionally include one or more of the following characteristics alone or in combination:
The metallic winding has an inductance ranging from 330 μH to 470 μH. Such inductance range allows to increase the measurement accuracy, more particularly when the distance between the transmitting and the receiving electrodes is about 20 mm or more.
The transmitting and the receiving electrodes are located at 3 mm at least from each other.
The transmitting and the receiving electrodes are substantially aligned according to their respective winding axis. This arrangement allows to improve the delivering, by the receiving electrode, through inductive coupling, of the second electric current.
The processor is configured to command the generation of the first electric current to the transmitting electrode at a frequency ranging from 20 KHz to 10 MHz, more preferably ranging from 70 KHz to 1 MHz and even more preferably ranging from 95 KHz to 110 KHz. Such frequency range allows to efficiently measure the intensity of the electric current induced in the receiving electrode and to determine the distance between the transmitting and the receiving electrodes with an accuracy of less than 200 μm and even of less than 5 μm while ensuring a resolution of the measurement of 3-5 μm.
The two movable elements are respectively a first half shell and a second half shell of a living bivalve mollusc.
The transmitting electrode and the receiving electrode are respectively fixed on the outside circumferential rim of the first half shell and of the second half shell so that said electrodes face each other, the transmitting electrode and the receiving electrode being adjacent to the hinge part of the living bivalve mollusc. Positioning the transmitting electrode and the receiving electrode in such part of the living bivalve mollusc allows to monitor precisely its growth over time.

According to another aspect, the invention can also relate to a method for determining a distance between two movable elements, the method being implemented in part by a processor, at least one pair of transmitting and receiving electrodes, the transmitting electrode and the receiving electrode being respectively fixed on the first and the second movable elements and located at less than 40 mm, more preferably less than 30 mm, from each other, said transmitting and receiving electrodes including at least a magnetic core and a metallic winding having an inductance ranging from 1 μH to 470 μH, the method comprising the following steps:
generating, by an energy source, a first electric current to the transmitting electrode, delivering, by the receiving electrode through inductive coupling, a second electric current,
converting, by the processor, the second electric current delivered by the receiving electrode, into at least one intensity value and generating at least one measurement signal which includes the intensity value,
carrying out a digital processing of the measurement signal and estimating, by the processor, the distance between the two movable elements from a mathematical law which links the distance between the two movable elements and the intensity value included in the measurement signal.

According to other optional features of the method according to the invention, it can optionally include one or more of the following characteristics alone or in combination:
the metallic winding has an inductance ranging from 330 μH to 470 μH.
The transmitting and the receiving electrodes are located at 3 mm at least from each other.
The transmitting and the receiving electrodes are substantially aligned according to their respective winding axis.
It comprises a step of commanding the generation of the first electric current to the transmitting electrode at a frequency ranging from 20 KHz to 10 MHz, more preferably ranging from 70 KHz to 1 MHz and even more preferably ranging from 95 KHz to 110 KHz.
   The two movable elements are respectively a first half shell and a second half shell of a living bivalve mollusc.
   The transmitting electrode and the receiving electrode are respectively fixed on the circumferential rim of the first half shell and the second half shell so that said electrodes face each other, the transmitting electrode and the receiving electrode being adjacent to the hinge part of the living bivalve mollusc.

According to another aspect, the invention also relates to a use of the system according to the invention to determine a growth of the living bivalve mollusc, the living bivalve mollusc growth being based on the estimate of the distance between the first half shell and the second half shell.

Optionally, the system may be used to determine a water pollution indicator based on the growth of the living bivalve mollusc.

### BRIEF DESCRIPTION OF THE DRAWINGS

The foregoing and other objects, features and advantages of the present invention will become more apparent from the following detailed description when taken in conjunction with the accompanying drawings in which:

FIG. 1 is a graphical illustration of a system for determining a distance according to the present invention.

FIG. 2 is a schematic view of a method for determining a distance according to the present invention.

Several aspects of the present invention are disclosed with reference to flow diagrams and/or block diagrams of methods, devices and computer program products according to embodiments of the invention.

On the figures, the flow diagrams and/or block diagrams show the architecture, the functionality and possible implementation of devices or systems or methods and computer program products, according to several embodiments of the invention.

For this purpose, each box in the flow diagrams or block diagrams may represent a system, a device, a module or code which comprises several executable instructions for implementing the specified logical function(s).

In some implementations, the functions associated with the box may appear in a different order than indicated in the drawings.

For example, two boxes successively shown, may be executed substantially simultaneously, or boxes may sometimes be executed in the reverse order, depending on the functionality involved.

Each box of flow diagrams or block diagrams and combinations of boxes in flow diagrams or block diagrams may be implemented by special systems that perform the specified functions or actions or perform combinations of special equipment and computer instructions.

### DETAILED DESCRIPTION

A description of example embodiments of the invention follows.

Below, we describe a summary of the invention and the associated vocabulary, before presenting the disadvantages of the prior art, then finally showing in more detail how the invention overcomes them.

By “processor” is meant, within the meaning of the invention, at least one hardware circuit configured to perform operations according to instructions contained in a code. The hardware circuit may be an integrated circuit. Examples of a processor include, but are not limited to, a central processing unit, a graphics processor, an application-specific integrated circuit (“ASIC” according to Anglo-Saxon terminology), and a programmable logic circuit. A single processor or several other units may be used to implement the invention.

By “coupled” is meant, within the meaning of the invention, connected, directly or indirectly, with one or more intermediate elements. Two elements may be coupled mechanically, electrically or linked by a communication channel.

By “computing device”, it should be understood any device comprising a processing unit or a processor, for example in the form of a microcontroller cooperating with a data memory, possibly a program memory, said memories possibly being dissociated. The processing unit cooperates with said memories by means of internal communication bus.

By “electrodes”, it should be understood any end of an electrical conductor adapted to release or capture an electric current, an electronic flow passing through a fluid or a vacuum.

The term “accuracy” is meant, within the meaning of the invention, the uncertainty, or the error, of a measurement value which characterizes the dispersion of values. More particularly, when the “accuracy” is expressed by a value, it should be understood in terms of an uncertainty range around a calculated value.

The invention proposes a system that allows to provide micro distances measurement between movable elements with a highly improved accuracy. In particular, the invention proposes to estimate a distance between two elements by measuring the intensity value of an electric current induced by a transmitting electrode to a receiving electrode comprising a metallic winding of a predetermined inductance. To that end, the invention proposes to convert the electric current induced into an intensity value from which the distance between the two movable elements can be inferred. The invention allows to provide a measurement accuracy of less than 200 μm, preferably of less than 100 μm and more preferably of less than 5 μm.

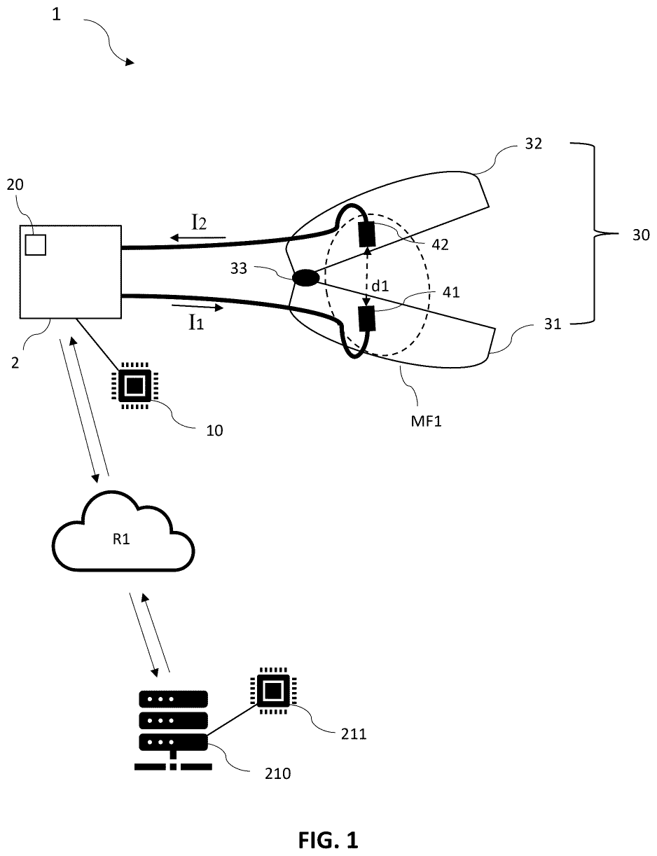

Thus, the invention relates to a system for determining a distance between two movable elements. As illustrated in FIG. 1, the system 1 comprises at least a processor 10, an energy source 20 and at least a pair of transmitting and receiving electrodes 41, 42.

In the system 1 for determining a distance d1 according to the invention, the transmitting and the receiving electrodes 41, 42 include at least a magnetic core and a metallic winding having an inductance ranging from 1 μH to 470 μH.

For instance, the magnetic core can be made of any material adapted to confine and guide magnetic fields. Any soft magnetic materials having high saturation magnetic flux density and high magnetic permeability can instead be used. More particularly, the magnetic core can be made of a ferromagnetic material alloy such as iron or ferrimagnetic compounds such as ferrites.

The metallic winding inductance can be adapted from 1 μH to 470 μH according to the distance between the two movable elements 31, 32 and consequently the distance between the electrodes. Moreover, the metallic winding inductance can also be adapted based on a predetermined measurement accuracy. Indeed, on the one hand the magnetic field generated by the transmitting electrode 41 must be adapted to induce the receiving electrode 42 to deliver an electric current according to said distance, and on the other hand the metallic winding inductance must be adapted to allow a predetermined measurement accuracy.

In a preferred embodiment of the system 1 according to the invention, the metallic winding has an inductance ranging from 330 μH to 470 μH.

More particularly, the transmitting electrode 41 and the receiving electrode 42 can correspond to an inductor coil.

In the system 1 according to the invention, the transmitting electrode 41 and the receiving electrode 42 are respectively fixed on the first and the second movable elements 31, 32.

Since the invention allows measuring micro distances by inductive coupling, the movable elements 31, 32 cannot be too far from each other. More particularly, the transmitting and the receiving electrodes 41, 42, which are fixed on the movable elements 31, 32, are located at less than 40 mm, more preferably less than 30 mm from each other. At this distance, the electronic noise contribution to the signal is minimal and the intensity value measured is characteristic of the distance d1 between the transmitting and the receiving electrodes 41, 42.

By way of illustrative examples, when the transmitting and the receiving electrodes 41, 42 comprise a metallic winding inductance of 470 μH, said electrodes are located at 30 mm from each other. Thus, the measurement accuracy is about 2 μm.

In an embodiment of the system 1 according to the invention, the transmitting and the receiving electrodes 41, 42 may substantially be aligned according to their respective winding axis.

As previously mentioned, the system 1 according to the invention for determining a distance d1 comprises an energy source 20. The energy source 20 is further configured to generate a first electric current I1 to the transmitting electrode 41 so that the receiving electrode 42 delivers, by inductive coupling, a second electric current I2. The energy source 20 may be a rechargeable or a non-rechargeable battery well-known from the skilled person.

As illustrated in FIG. 1, the transmitting electrode 41 and the receiving electrodes 42 can be coupled, through dedicated wires, to a computing device 2 housing the energy source 20 and the processor 10. The processor 10 may be configured to command the generation, by the energy source 20, of the first electric current I1 to the transmitting electrode 41. The transmitting electrode 41 generates a magnetic field MF1 (based on the magnetic core and the electric current I1) and the receiving electrode 42 receives the magnetic field MF1. Thus, the receiving electrode 42 delivers a second electric current I2, the intensity of which depends on the one hand of the distance between the transmitting and the receiving electrodes 41, 42 and on the other hand on the intensity of the first electric current I1. More particularly, when the distance d1 between the transmitting electrode 41 and the receiving electrode 42 changes because of the movable elements 31, 32 moving towards or away from each other, the intensity of the second electric current I2 will increase or decrease respectively.

In an embodiment of the system 1 according to the invention, the processor 10 is configured to command the generation of the first electric current I1 to the transmitting electrode 41 at a frequency ranging from 20 KHz to 10 MHz, more preferably ranging from 70 KHz to 1 MHz and even more preferably ranging from 95 KHz to 110 KHz.

The processor 10 controls the generation of the first electric current I1 which supplies the transmitting electrode 41, with alternating current, at a predetermined frequency, such as those mentioned previously. According to the invention, the processor 10 is configured to convert the electric current I2 delivered by the receiving electrode 42, into at least one intensity value and to generate at least one measurement signal which includes said intensity value.

The intensity of the second electric current I2 induced in the receiving electrodes 42, can be determined by a sensor adapted to measure the intensity of the second electric current I2. The system 1 according to the invention can thus comprise a converter of an analog signal generated by the sensor into a digital signal.

In an embodiment of the system 1, the processor 10 can be configured to sample the measurement signal at a frequency ranging from 20 KHz to 10 MHz, more preferably ranging from 70 KHz to 1 MHz and even more preferably ranging from 95 KHz to 110 KHz.

For example, a measurement signal, for each pair of transmitting/receiving electrodes 41, 42, may be generated in a sequential order every 0.1 s. The measurement signal may comprise three packets of information including: an intensity value representative of the electric current I2 delivered by the receiving electrode 42, a timestamp of the measurement signal, and a value related to the pair of transmitting/receiving electrodes 41, 42 from which the measurement signal is generated.

In the system 1 according to the invention, the processor 10 is further configured to carry out a digital processing of the measurement signal to obtain an estimate of the distance between the two movable elements 31, 32 from a mathematical law which links the distance between the two movable elements 31, 32 and the intensity value included in the measurement signal.

In addition, the system 1 may comprise a remote server 210 including a second processor 211. The processor 10 can be configured to send the estimate of the distance d1, by a dedicated communication module, to the remote server 210.

Alternatively, the processor 10 can be configured to send the measurement signals, by a dedicated communication module, to the remote server 210. In that case, the second processor 211 of the remote server 210 may be configured to carry out the digital processing of the measurement signal to obtain an estimate of the distance between the two movable elements 31, 32 from a mathematical law which links the distance between the two movable elements 31, 32 and the intensity value included in the measurement signal.

The estimate of the distance d1 or the measurement signals can be sent through a wire or wireless communication bus R1 of known type, for example through Wi-Fi®, Bluetooth®, ZigBee®, SigFox®, LoRaWan®, GPRS, UMTS, LTE, WiMax® or NB-IOT protocols.

The system 1 according to the invention may further comprise additional sensors, such as physico-chemical detectors (hydrocarbon detector in water for example), automatic water samplers, turbidity probes, pH probes, chlorophyl A probes, temperature probes or dissolved O₂ probes. The additional sensors may include a hardware and software arrangement configured to send the measurements to the remote server 210 or said additional sensors can be coupled to the computing device 2 which can be configured to send the additional sensor measurements to the remote server 210.

To calibrate the distance measurement, the processor 10 or the second processor 211 may use one or more measurement signal(s) related to a predetermined inductance of the transmitting and the receiving electrodes 41, 42 and a predetermined distance between said electrodes to determine a mathematical law which links the intensity variation between the first electric current I1 and the second electric current I2 and the distance between said electrodes.

In particular, the mathematical law can be determined from a first state where the distance between the movable elements 31, 32 is substantially zero. In this case, the variation in the measured intensity values is considered as a variation from 0% to 100% of a predetermined maximum distance, for example 30 mm or 40 mm, between the movable elements 31, 32.

In a preferred embodiment, the system 1 according to the invention may provide a measurement accuracy of less than 3 μm. To that end, the pair of transmitting and receiving electrodes 41, 42, are being respectively fixed on the first and the second movable elements 31, 32 at 30 mm from one electrode to another. The pair of transmitting and receiving electrodes 41, 42 comprise a magnetic core and a metallic winding having an inductance ranging from 330 μH to 470 μH. More particularly, the metallic winding of both electrodes may have an inductance of 470 μH.

The system according to the invention is particularly suited to monitor growth of a biosensor, more particularly a daily growth of a living bivalve mollusc 30.

Indeed, a solution considered for monitoring the quality of an environment is to measure the physical characteristics of a biosensor and more particularly its growth rate. Indeed, in a healthy environment, some biosensors will show regular growth unlike a biosensor living in an environment degraded by harmful substances. In addition, it may happen that the evolution of the growth rate of a biosensor is so small that it needs to be determined by a high-precision measuring instrument to be able to be measured in real time. However, the measuring instruments disclosed in the prior art have measurement accuracy that are too low to detect the growth rate of some of these biosensors within a sufficiently short period of time to be considered as “in real time”.

In an embodiment of the system 1 according to the invention, the two movable elements 31, 32 may respectively be a first half shell and a second half shell of a living bivalve mollusc 30.

More particularly, in a preferred embodiment of the system 1, the transmitting electrode 41 and the receiving electrode 42 may respectively be fixed on the outside circumferential rim of the first half shell and of the second half shell so that said electrodes 41, 42 face each other, the transmitting electrode 41 and the receiving electrode 42 being adjacent to the hinge part 33 of the living bivalve mollusc 30.

In a preferred embodiment, to limit the size of the transmitting and the receiving electrodes 41, 42, each of said electrodes can weigh less than 1 g (out of water), more particularly less than 0.6 g. Such weigh is also very advantageous when the electrodes are used on a living bivalve mollusc 30 since it is light enough not to interfere with normal behaviour of the living bivalve mollusc 30. Data collected from the electrodes are thus particularly relevant for characterizing the living bivalve mollusc activity.

In another embodiment of the system 1 according to the invention, the transmitting electrode 41 and the receiving electrode 42 are made up of two resin-coated and are glued on to each of the movable elements 31, 32. The two resin-coated are adapted to prevent any physical interaction of the electrodes with the external environment, more particularly to be waterproof and resistant chemical compounds composing fresh water and/or seawater.

According to a second aspect, the invention relates to a use of a system according to the invention to determine a growth of the living bivalve mollusc 30, the living bivalve mollusc growth being based on the estimate of the distance d1, measured daily, between the first half shell and the second half shell.

In an optional embodiment of the use of a system 1 according to the invention, a water pollution indicator may be determined based on the growth of the living bivalve mollusc 30.

Indeed, when the two movable elements 31, 32 are respectively a first half shell and a second half shell of a living bivalve mollusc 30, the system 1 according to the invention allows to determine the growth rate of the living bivalve mollusc 30. The growth rate is obtained by selection of the minimal daily opening value of the living bivalve mollusc 30; a change in growth rates of the living bivalve mollusc 30 can reflect change in natural conditions, for example water temperature or exposure to harmful stressors. The growth rate is characterized by the distance between the transmitting electrode 41 and the receiving electrode 42 which increases daily of approximatively 5 μm to 25 μm for bivalve molluscs of the Ostreidae family in standard conditions.

It is well-known that oysters and other molluscs, such as clams, scallops, mussels, etc. obtain the nutrients they need from the water, the shells open in order to filter out the food particles, i.e. small algae, zooplankton, and at other times the bivalve (two-part) shell is either open less wide or is fully closed following precise environmental timing. This natural behaviour can be used as an indicator of the quality of the water in which molluscs live.

The use of the system 1 according to the invention allows to reconstruct natural behaviour of bivalves such as: circadian and circatidal cycles, spawning events, growth rates including growth arrest and acceleration and death.

Thus, the system 1 according to the invention can be used as a bio-sensor tool for in-situ, continuous and bulk indication of water quality around industrial facilities (marine, brackish and fresh waters) to monitor water quality and to detect changes possibly due to anthropogenic/industrial activities at early stage in order to take appropriate mitigation measures.

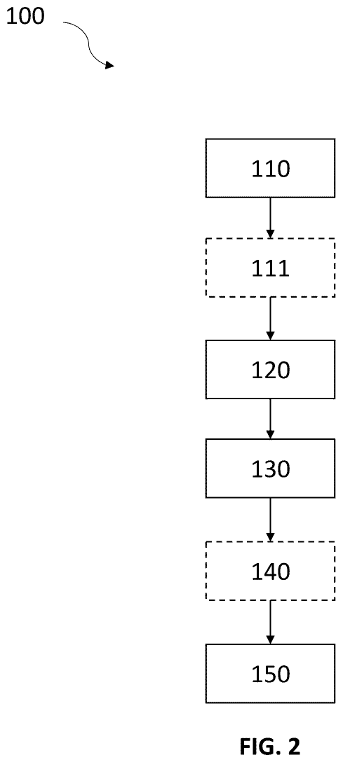

According to a third aspect, as shown in FIG. 2, the invention relates to a method for determining 100 a distance d1 between two movable elements 31, 32, the method 100 being implemented in part by a processor 10, at least one pair of transmitting and receiving electrodes 41, 42, the transmitting electrode and the receiving electrodes 41, 42 being respectively fixed on the first and the second movable elements 31, 32 and located at less than 40 mm, more preferably less than 30 mm, from each other, said transmitting and receiving electrodes 41, 42 including at least a magnetic core and a metallic winding having an inductance ranging from 1 μH to 470 μH, the method 100 comprising the following steps:
generating 110, by an energy source 20, a first electric current I1 to the transmitting electrode 41,
delivering 120, by the receiving electrode 42 through inductive coupling, a second electric current I2,
converting 130, by the processor 10, the second electric current I2 delivered by the receiving electrode 42, into at least one intensity value and generating at least one measurement signal which includes the intensity value,
carrying outb 150 a digital processing of the measurement signal and estimating, by the processor 10, the distance d1 between the two movable elements 31, 32 from a mathematical law which links the distance between the two movable elements 31, 32 and the intensity value included in the measurement signal.

The method 100 according to the invention allows to provide a measurement accuracy of less than 200 μm, preferably of less than 100 μm and more preferably of less than 5 μm.

The method 100 according to the invention may optionally comprise a step of commanding 111 the generation of the first electric current I1 to the transmitting electrode 41 at a frequency ranging from 20 KHz to 10 MHz, more preferably ranging from 70 KHz to 1 MHz and even more preferably ranging from 95 KHz to 110 KHz.

The method 100 according to the invention may optionally comprise, before the step of carrying out 150 a digital processing of the measurement signal, a step of sampling 140 the analog signal at a frequency ranging from 20 KHz to 10 MHz, more preferably ranging from 70 KHz to 1 MHz and even more preferably ranging from 95 KHz to 110 KHz. The step of sampling 140 the analog signal can implement sampling techniques well-known by the skilled person.

The method 100 for determining a distance may be implemented by a system 1 according to the invention.

The invention can be the subject of numerous variants and applications other than those described above. In particular, unless otherwise indicated, the different structural and functional characteristics of each of the implementations described above should not be considered as combined and/or closely and/or inextricably linked to each other, but on the contrary as simple juxtapositions. In addition, the structural and/or functional characteristics of the various embodiments described above may be the subject in whole or in part of any different juxtaposition or any different combination.

1. A system for determining a distance between two movable elements the system comprising a processor, an energy source and transmitting electrode and a receiving electrodes, the transmitting electrode and the receiving electrode being respectively fixed on the two movable elements, the energy source being further configured to generate a first electric current to the transmitting electrode so that the receiving electrode delivers, by inductive coupling, a second electric current in which:
the transmitting electrode and the receiving electrodes are located less than 40 mm from each other and each include
a magnetic core,
a metallic winding having an inductance ranging from 1 μH to 470 μH,
the processor is configured to convert the electric current delivered by the receiving electrode into an intensity value and to generate a measurement signal which includes said intensity value,
the processor is further configured to carry out a digital processing of the measurement signal to obtain an estimate of the distance between the two movable elements, from a mathematical law which links the distance between the two movable elements and the intensity value included in the measurement signal.

2. The system according to claim 1, wherein the metallic winding has an inductance ranging from 330 μH to 470 μH.

3. The system according to claim 1, wherein the transmitting electrode and the receiving electrodes are located at least 3 mm from each other.

4. The system according to claim 1, wherein the transmitting electrode and the receiving electrodes are substantially aligned according to their respective winding axis.

5. The system according to claim 1, wherein the processor is configured to command the generation of the first electric current to the transmitting electrode at a frequency ranging from 20 KHz to 10 MHz.

6. The system according to anyone of claim 1, wherein the two movable elements are respectively a first half shell and a second half shell of a living bivalve mollusc

7. The system according to claim 6, wherein the transmitting electrode and the receiving electrode are respectively fixed on an outside circumferential rim of the first half shell and of the second half shell so that said electrodes face each other, the transmitting electrode and the receiving electrode being adjacent to a hinge part of the living bivalve mollusc.

8. A method for determining a distance between two movable elements, the method being implemented in part by a processor, a transmitting electrode and a receiving electrode the transmitting electrode and the receiving electrodes being respectively fixed on the first and the second movable elements and located less than 40 mm, from each other, said transmitting and receiving electrodes each including a magnetic core and a metallic winding having an inductance ranging from 1 μH to 470 μH, the method comprising the following steps:
generating, by an energy source, a first electric current to the transmitting electrode,
delivering, by the receiving electrode through inductive coupling, a second electric current,
converting, by the processor, the second electric current delivered by the receiving electrode, into an intensity value and generating a measurement signal which includes the intensity value,
carrying out a digital processing of the measurement signal and estimating, by the processor, the distance between the two movable elements from a mathematical law which links the distance between the two movable elements and the intensity value included in the measurement signal.

9. The method according to claim 8, wherein the metallic winding has an inductance ranging from 330 μH to 470 μH.

10. The method according to claim 8, wherein the transmitting electrode and the receiving electrodes are located at least 3 mm from each other.

11. The method according to claim 8, wherein the transmitting electrode and the receiving electrodes are substantially aligned according to their respective winding axis.

12. The method according to claim 8, further comprising a step of commanding the generation of the first electric current to the transmitting electrode at a frequency ranging from 20 KHz to 10 MHz.

13. The method according to claim 8, wherein the two movable elements are respectively a first half shell and a second half shell of a living bivalve mollusc.

14. The method according to claim 13, wherein the transmitting electrode and the receiving electrode are respectively fixed on circumferential rims of the first half shell and the second half shell so that said electrodes face each other, the transmitting electrode and the receiving electrode being adjacent to a hinge part of the living bivalve mollusc

15. A method of determining growth of a living bivalve mollusc, based on the estimate of the distance between the first half shell and the second half shell of the system according to claim 6.

16. The method according to claim 15, wherein a water pollution indicator is determined based on the growth of the living bivalve mollusc.

17. The system according to claim 1, said transmitting electrode and receiving electrode being located less than 30 mm from each other.

18. The system according to claim 5, said frequency ranging from 95 KHz to 110 KHz.
